(19) United States
(12) Patent Application Publication
(10) Pub. No.: US 2017/0112560 A1
(43) Pub. Date: Apr. 27, 2017

(54) SURGICAL INSTRUMENTS WITH COOLING ASSEMBLY

(71) Applicant: COVIDIEN LP, Mansfield, MA (US)

(72) Inventors: ANTHONY B. ROSS, BOULDER, CO (US); WILLIAM H. NAU, JR., LONGMONT, CO (US); ARLEN K. WARD, ENGLEWOOD, CO (US); DUANE E. KERR, LOVELAND, CO (US); WILLIAM ROSS WHITNEY, BOULDER, CO (US); CASSANDRA LATIMER, THORNTON, CO (US)

(21) Appl. No.: 15/232,074

(22) Filed: Aug. 9, 2016

Related U.S. Application Data
(60) Provisional application No. 62/244,227, filed on Oct. 21, 2015.

(51) Int. Cl. A61B 18/08; A61B 18/14; A61B 18/04

(52) U.S. Cl. CPC A61B 18/085; A61B 18/04; A61B 18/1445; A61B 2018/00011

(57) ABSTRACT

A cooling assembly for use with an electrosurgical instrument is provided. The cooling assembly includes a body, and a cavity. At least a portion of an inner perimeter of the body is configured for selective engagement with a jaw member of the electrosurgical instrument. The cavity is defined within the body and configured to contain a fluid therein. The fluid is capable of at least one of cooling or thermal insulation to areas laterally adjacent the jaw member.

### CROSS REFERENCE TO RELATED APPLICATION

The present application claims the benefit of and priority to U.S. Provisional Application Ser. No. 62/244,227, filed on Oct. 21, 2015, the entire contents of which are incorporated herein by reference.

### BACKGROUND

Technical Field

The present disclosure relates to surgical instruments and, more particularly, to surgical instruments including jaw members for grasping, treating, sealing, stapling, and/or dividing tissue, and where the jaw members include a cooling assembly.

Description of Related Art

Many surgical instruments are known for sealing, stapling, or otherwise joining tissue. Some of these surgical include one or more movable handles, levers, actuators, triggers, etc. for actuating and/or manipulating one or more functional components of the surgical instrument. For example, a surgical forceps may include a movable handle that is selectively actuatable relative to a stationary handle for moving at least one jaw member with respect to another jaw member of the forceps between spaced-apart and approximated positions for grasping tissue therebetween. Such a forceps may further include additional triggers for selectively actuating electrosurgical energy or for deploying staples, and/or for deploying a knife between the jaw members to cut tissue grasped therebetween.

In certain types of surgical procedures, it may be useful to control thermal spread from sealing devices to surrounding tissue. Accordingly, a surgical instrument including capabilities to cool its jaw members and thus help limit thermal spread may be useful.

### SUMMARY

The present disclosure relates to a cooling assembly for use with an electrosurgical instrument. The cooling assembly includes a body and a cavity. At least a portion of an inner perimeter of the body is configured for selective engagement with a jaw member of the electrosurgical instrument. The cavity is defined within the body and is configured to contain a fluid therein. The fluid is capable of providing cooling and/or thermal insulation to areas laterally adjacent the jaw member.

In aspects of the present disclosure, the fluid is passively cooled or actively cooled.

In other aspects, the fluid includes expandable gas and/or alcohol, and that the body includes silicone.

In yet other aspects, the body may be U-shaped.

In still other aspects, the fluid may be configured to provide cooling and/or thermal insulation to areas distally adjacent the jaw member(s).

The present disclosure also relates to a surgical instrument including a handle assembly, an elongated shaft, an end effector, and a cooling assembly. The elongated shaft extends distally from the handle assembly. The end effector is engaged to a distal portion of the elongated shaft and includes a first jaw member and a second jaw member. At least one of the first and second jaw members is movable with respect to the other jaw member to grasp tissue therebetween. The cooling assembly is disposed in mechanical cooperation with the first jaw member and includes a deployable reservoir configured to house a fluid therein. The cooling assembly is configured to provide cooling and/or thermal insulation to areas laterally adjacent the first jaw member.

In aspects, the cooling assembly is disposed in mechanical cooperation with the second jaw member and is configured to provide cooling and/or thermal insulation to areas laterally adjacent the first and/or second jaw member. The fluid may be passively cooled or actively cooled, and may expandable gas and/or alcohol.

In other aspects, the deployable reservoir is U-shaped.

In aspects, a tissue-contacting surface of the first jaw member defines a plane, and an entirety of the deployable reservoir is spaced from the plane. The cooling assembly may be configured to overlap an intersection between a lateral edge and a tissue-contacting surface of the first jaw member.

In yet other aspects, the deployable reservoir is expandable from a first position wherein the deployable reservoir includes a first amount of fluid therein, to a second position wherein the deployable reservoir includes a second amount of fluid therein. In the first position, the deployable reservoir may be undeployed, and in the second position, the deployable reservoir may be deployed. The second amount of fluid may be greater than the first amount of fluid.

### BRIEF DESCRIPTION OF THE DRAWINGS

Various aspects of the present disclosure are described herein with reference to the drawings wherein like reference numerals identify similar or identical elements:

FIG. 1 is a perspective view of an embodiment of a surgical instrument in accordance with the present disclosure;

FIG. 2A is an enlarged perspective view of an end effector of the surgical instrument of FIG. 1 with the jaw members in an open position and depicting a cooling assembly in a undeployed configuration;

FIG. 2B is a perspective view of the end effector of FIG. 2A depicting the cooling assembly in a deployed configuration;

FIGS. 2C and 2D are perspective views of the end effector of FIGS. 2A and 2B depicting the cooling assembly in alternate deployed configurations;

FIG. 3 is a plan view of a cooling assembly of a first jaw member of the end effector FIG. 2B;

FIG. 4 is a plan view of an alternate embodiment of a cooling assembly of the first jaw member in accordance with the present disclosure;

FIG. 5 is a partial, transverse cross-sectional view of the cooling assembly on the first jaw member of FIG. 3;

FIG. 6 is a partial, transverse cross-sectional view of an alternate embodiment of a cooling assembly on the first jaw member in accordance with the present disclosure;

FIG. 7 is a top view of an alternate embodiment of a cooling assembly;

FIG. 8 is a perspective view of the cooling assembly of FIG. 7 engaged with the first jaw member of an end effector; and

FIG. 9 is a schematic illustration of a surgical system in accordance with the present disclosure.

### DETAILED DESCRIPTION

Embodiments of the presently disclosed surgical instrument are described in detail with reference to the drawings, in which like reference numerals designate identical or corresponding elements in each of the several views. As used herein the term “distal” refers to that portion of the surgical instrument that is farther from the user, while the term “proximal” refers to that portion of the surgical instrument that is closer to the user.

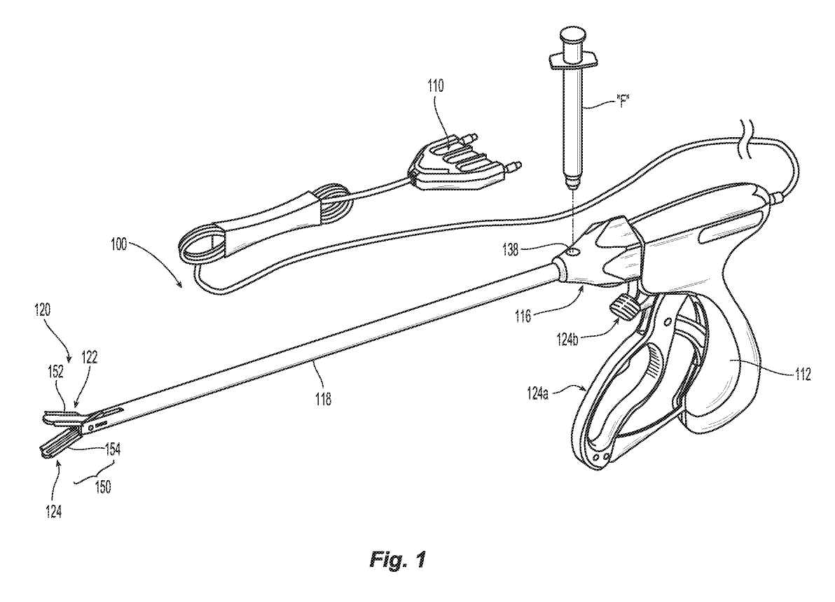

Referring initially to FIG. 1, an embodiment of a surgical instrument 100 is shown for use with various surgical procedures. Surgical instrument 100 may be configured to connect to a source of electrosurgical energy (not shown) via connector assembly 110, and/or may contain an independent energy source e.g., a battery (not shown). The use of an electrosurgical apparatus to apply electrosurgical energy to tissue is generally described in U.S. Pat. No. 7,083,618, which is incorporated herein in its entirety by reference.

Surgical instrument 100 includes a handle assembly 112 near a proximal end, an end effector 120 near a distal end and an elongated shaft 118 extending therebetween. The end effector 120 includes a first jaw member 122 and a second jaw member 124, which are movable relative to each other. The end effector 120 may be positioned within a body cavity to engage tissue at a surgical site while handle assembly 112 is manipulatable by a surgeon from outside the body cavity to control the movement and operation of the end effector 120. Handle assembly 112 includes a movable handle 124a, which is manipulatable to open and close the end effector 120, and a trigger 124b, which is manipulatable to initiate an electrosurgical current.

Actuation of the movable handle 124a applies a force to the jaw members 122 and 124, in one embodiment, in the range of about 3 kg/cm² to about 16 kg/cm². Also, in the closed or approximated configuration, a separation or gap distance is maintained between the jaw members 122, 124 by an array of stop members (not shown). In some embodiments, to provide an effective tissue seal, an appropriate gap distance of between about 0.001 inches to about 0.006 inches may be provided. Further, details of a vessel sealing device including a handle assembly for controlling actuation of an end effector can be found in U.S. Pat. Nos. 7,101,371 and 7,083,618, which are incorporated herein in their entirety by reference.

The end effector 120 also includes a cooling assembly 150 configured to provide cooling and/or to thermally insulate at least a portion of the jaw members 122, 124 (e.g., at least a portion of a perimeter of each jaw member 122, 124) to help protect adjacent tissue during a surgical procedure (e.g., sealing of tissue), as discussed in further detail below. The cooling assembly 150 can actively or passively provide a cooling or thermally insulating effect on the jaw members 122, 124 and/or adjacent tissue.

In embodiments where jaw members 122, 124 are actively cooled or thermally insulated, a fluid conduit 138 extends from the handle assembly 112, through the elongated shaft 118, and to the end effector 120. Thus, a fluid may be injected into the fluid conduit 138 from a fluid source “F” (FIG. 1) from outside the body to supply a cooling or a thermally insulating fluid and/or to deploy the cooling assembly 150 within a body cavity. Alternatively, the fluid source “F” may be included in a reservoir within a portion of the surgical instrument 100 (e.g., within handle assembly 112).

The cooling or insulating fluid may be any suitable fluid (inclusive of liquids, air, and gels) including, but not limited to air, helium, water, saline, carbon dioxide, nitrous oxide, ethylene glycol, mineral oil, polyalkylene glycol, refrigerants, and nanofluids. Additionally, active cooling of the jaw members 122, 124 may take place via the Peltier Effect by applying voltage between two electrodes connected to a semiconductor material to create a temperature difference. Passive cooling of the jaw members 122, 124 may also be accomplished by encapsulating a fluid (inclusive of liquids, air, and gels) within an alternative cooling assembly 250, as described in further detail below.

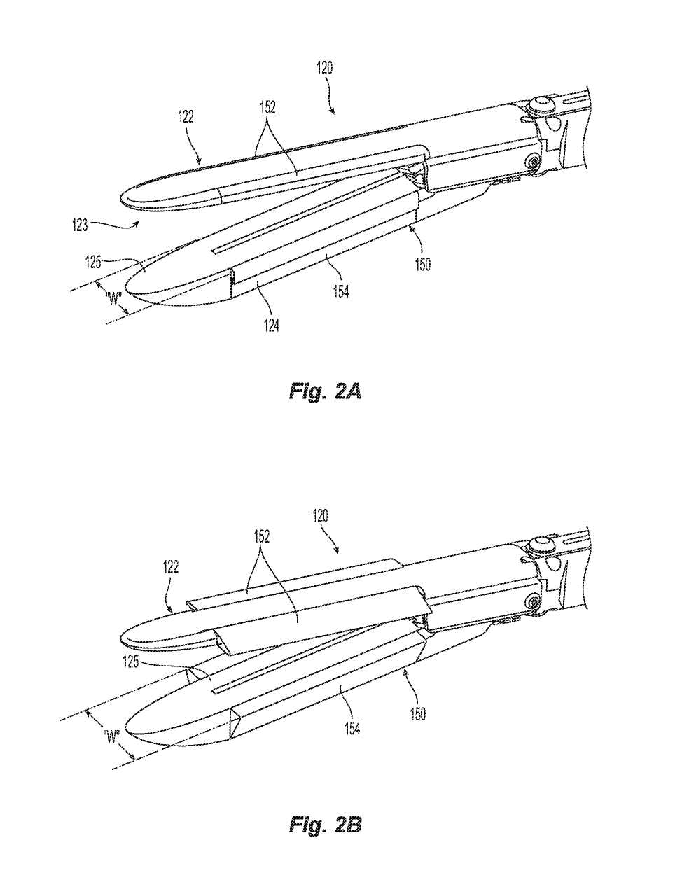

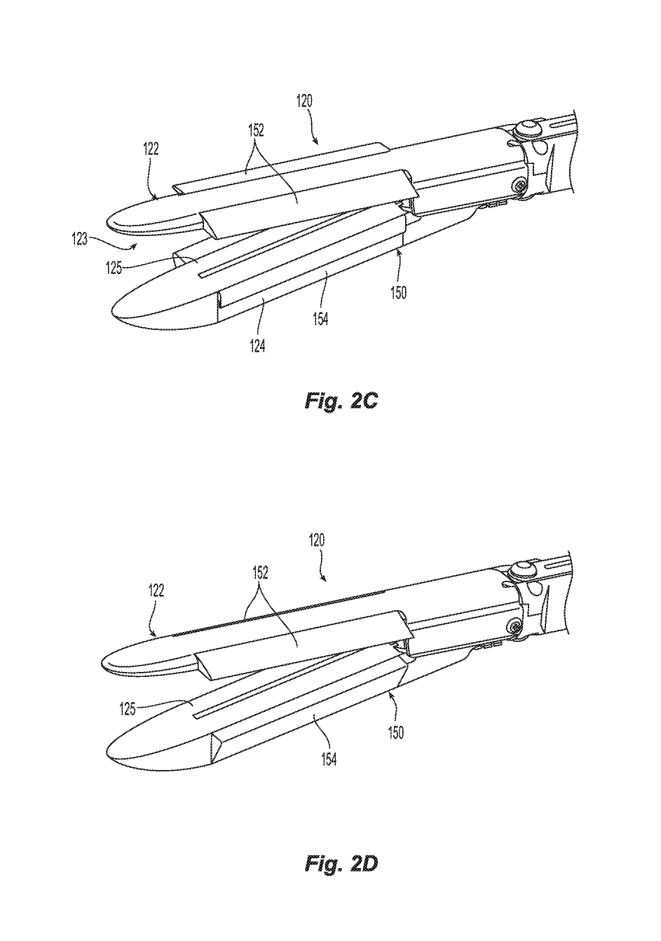

Referring now to FIG. 2A, end effector 120 is depicted with the jaw members 122, 124 substantially spaced from one another. In this position, tissue may be inserted between a tissue-contacting surface 123 of the first jaw member 122, and a tissue-contacting surface 125 of the second jaw member 124. The tissue may be clamped to an appropriate thickness by approximating the jaw members 122, 124 relative to each other, and thereafter, electrosurgical energy may be used to seal the tissue. In FIG. 2A, the cooling assembly 150 is in an undeployed or uninflated position. When a surgeon anticipates that the heat of the jaw members 122, 124 (experienced during sealing tissue, for example) may undesirably affect tissue that is adjacent at least one jaw member 122 or 124, the surgeon may deploy or activate the cooling assembly 150. The cooling assembly 150 may automatically deploy as well, as described below.

The cooling assembly 150 includes a pair of reservoirs 152, 154 in fluid communication with the fluid conduit 138, and disposed adjacent jaw members 122, 124. The reservoirs 152, 154 are deployed by filling them with the fluid such that the reservoirs 152, 154 extend laterally from lateral surfaces of the jaw members 122 and 124. The reservoirs 152, 154 may be selectively deployed such that they extend distally from distal surfaces of the jaw members 122 and 124. The reservoirs 152, 154 of the cooling assembly 150 are retracted or collapsed by removing the fluid from the reservoirs 152, 154.

As depicted in FIG. 2A, the reservoirs 152, 154 may be maintained in an undeployed condition such that a lateral width “W” of the end effector 120 is minimized. Minimizing the lateral width “W” tends to facilitate insertion of the end effector 120 into a body cavity through a cannula, for example, and may also provide maneuverability in the body cavity to facilitate proper positioning of the end effector 120 adjacent the targeted tissue.

The cooling assembly 150 may be selectively deployed to the configuration depicted in FIG. 2B once the targeted tissue is positioned between the jaw members 122, 124, or at any other time the surgeon deems appropriate. When deployed, the first pair of reservoirs 152 extends laterally from the first jaw member 122, and the second pair of reservoirs 154 extends laterally from the second jaw member 124.

The reservoirs 152, 154 are configured for collective deployment such that each of the reservoirs 152, 154 is deployed concurrently upon a single actuation by the surgeon at the handle assembly 112. For instance, each of the reservoirs 152, 154 fluidly communicates with the fluid conduit 138 such that injection of fluid through the fluid conduit 138 fills each of the reservoirs 152, 154 concurrently. Alternatively, the reservoirs 152, 154 may be configured for individual deployment or deployment in pairs. For example, the first pair of reservoirs 152 may fluidly communicate with the fluid conduit 138 while the second pair of reservoirs 154 fluidly communicates with an additional fluid conduit (not shown). This configuration offers an additional degree of control to a surgeon. For example, a surgeon may deploy reservoirs 152 while maintaining reservoirs 154 in an undeployed configuration as depicted in FIG. 2C. The reservoirs 152, 154 may be configured to permit deployment of the reservoirs 152, 154 on one lateral side of the end effector 120 while maintaining the reservoirs 152, 154 on another lateral side of the end effector 120 in an undeployed configuration as depicted in FIG. 2D. The reservoirs 152, 154 may be selectively deployed as desired based on the location of the adjacent tissue to be protected from the heat of the jaw members 122, 124.

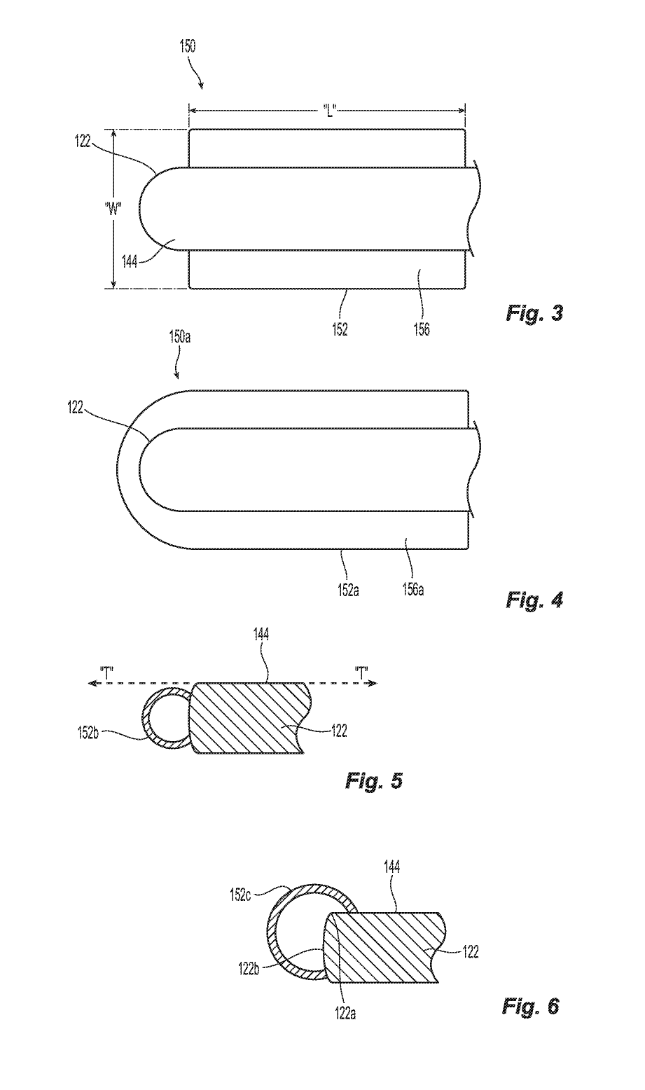

Referring now to FIG. 3, the first pair of fluid reservoirs 152 extends laterally on opposing sides of a tissue-contacting surface 144 to define the first pair of cooling members 156. The cooling members 156 extend in a lateral direction to define the lateral width “W” as described above, and extend in a longitudinal direction so as to have a sufficient length “L” to flank a majority of the length of jaw members 122, 124 (first jaw member 122 is shown in FIG. 3), for example.

FIG. 4 depicts an alternate embodiment of a cooling assembly 150a. A reservoir 152a extends continuously along both longitudinal sides of the first jaw member 122, and also along the distal end of the first jaw member 122. A cooling member 156a thus defines a generally U-shaped perimeter around the first jaw member 122. A similar cooling assembly (not shown) may be arranged around the second jaw member 124. Further, cooling member 156a may be dimensioned to follow the perimeter of any shaped jaw member, including linear jaws (e.g., having a dolphin tip or a blunt tip), curved jaws, etc.

Referring now to FIGS. 5 and 6, cross-sectional views of different embodiments of reservoir 152 are shown in connection with a portion of the first jaw member 122. In FIG. 5, reservoir 152b is disposed adjacent the first jaw member 122 and extends laterally therefrom without overlapping the tissue-contacting surface 144. Moreover, an entirety of the reservoir 152b is spaced from (e.g., below) a plane “T-T” defined by the tissue-contacting surface 144 of the first jaw member 122.

In FIG. 6, reservoir 152c is disposed adjacent the first jaw member 122, extends laterally therefrom, and overlaps the tissue-contacting surface 144 (e.g., the intersection 122a between tissue-contacting surface 144 and a lateral edge 122b of the first jaw member). Reservoir 152b and/or reservoir 152c are usable with each jaw member 122, 124 and with any of the embodiments disclosed herein.

The cooling members 156 disclosed herein may be collapsed or retracted by evacuating and deflating the reservoirs 152, 154. Fluid may be actively withdrawn by generating a reduced pressure at the fluid source “F” (FIG. 1). In this manner, the fluid is returned to the fluid source “F” under the influence of a suction force. Alternatively, the reservoirs 152, 154 may be evacuated by establishing a fluid flow path between the reservoirs 152, 154 and the atmosphere, thus permitting the fluid to vent. Retracting the reservoirs 152, 154 facilitates removal of the surgical instrument 100 through a cannula, for example.

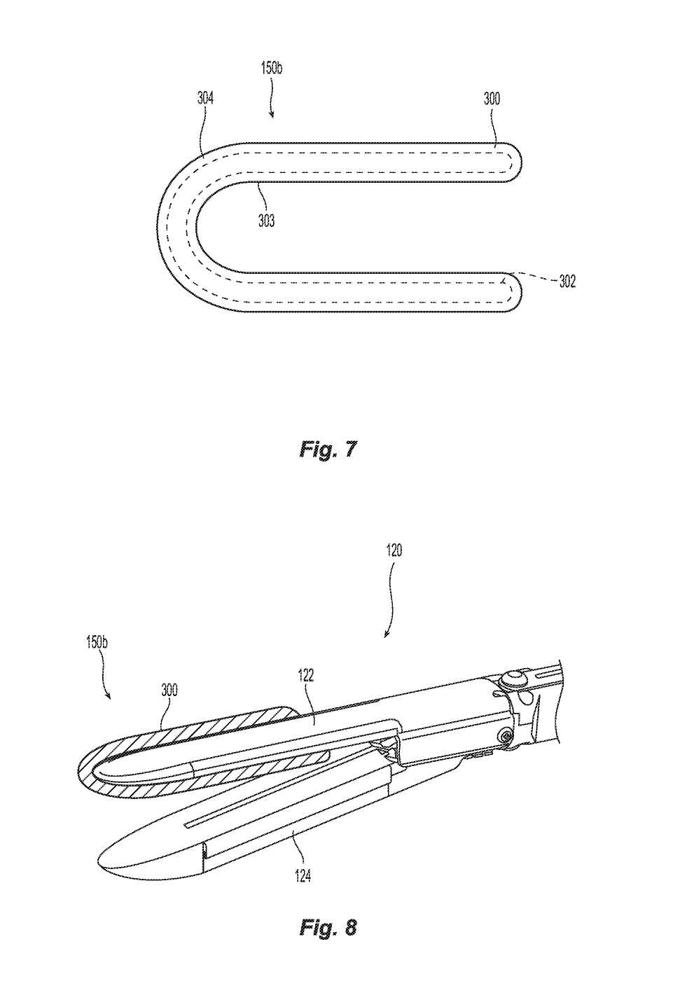

FIGS. 7 and 8 depict an alternate embodiment of a cooling assembly 150b including a bumper 300. FIG. 7 illustrates the bumper 300 on its own, and FIG. 8 illustrates the bumper 300 engaged with the first jaw member 122 of end effector 120. Cooling assembly 150b includes one bumper 300 or engagement member for each jaw member 122, 124. That is, a first engagement member or bumper 300 is selectively engageable with the first jaw member 122, and a second engagement member or bumper 300 is selectively engageable with the second jaw member 124. Bumper 300 is selectively attachable to a jaw member 122, 124 and is configured to provide thermal insulation between the jaw member 122, 124 and surrounding tissue. More particularly, an inner perimeter 303 of a body 304 of bumper 300 is selectively engageable with jaw member 122, 124 by known mechanical elements or mechanisms (e.g., a snap-fit engagement of two complimentary parts) and/or with the use of an adhesive, for example.

Bumper 300 may be actively cooled, as described above with regard to cooling assemblies 150 and 150a, or bumper 300 may be passively cooled. In embodiments where bumper is passively cooled, a cavity 302 within body 304 of bumper 300 is partially filled with a gas (e.g., expanding gas), a fluid (e.g., alcohol), or a gel. The body 304 of the bumper 300 surrounds the cavity 302 and may be made from silicone and/or another insulating material. In either the active or passing cooling embodiments, bumper 300 may include a port extending through body 304 and in fluid engagement with cavity 302 to enable the gas, fluid or gel, for instance, to enter and exit the cavity 302.

Any of the reservoirs 152, 154 or the body 304 of the bumper 300 of the present disclosure may include a wound treatment material. The wound treatment material may include an adhesive sealant, a hemostat such as a fibrin based material or a medicament such as a drug, enzyme, growth factor or a diagnostic agent. Many other possible wound treatment materials are described in U.S. Pat. No. 7,455,682, which is incorporated by reference herein in its entirety.

The present disclosure also relates to methods of joining (e.g., sealing, fastening, etc.) tissue while protecting adjacent tissue by providing cooling and/or thermal insulation. The methods include using surgical instrument 100 with cooling assemblies 150, 150a, and/or 150b, as described above. The methods may include inserting the jaw members 122, 124 into a surgical site, deploying reservoirs 152, 154 by filling them with a fluid, using the jaw members 122, 124 to seal or fuse tissue within the surgical site, deflating the reservoirs 152, 154 by removing the fluid, and removing the jaw members 122, 124 from the surgical site.

The methods may also include engaging bumper 300 with the first jaw member 122 and/or the second jaw member 124, inserting the jaw members 122, 124 into a surgical site, using the jaw members 122, 124 to seal or fuse tissue within the surgical site, removing the jaw members 122, 124 from the surgical site, and removing the bumper 300 from the jaw member(s) 122, 124.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prepare the patient for surgery and configure the robotic surgical system with one or more of the surgical instruments disclosed herein while another surgeon (or group of surgeons) remotely controls the instrument(s) via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

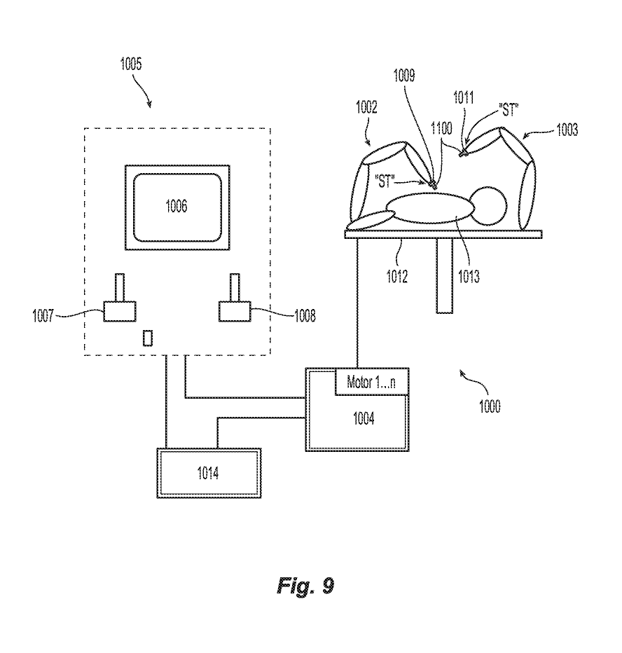

With particular reference to FIG. 9, a medical work station is shown generally as work station 1000 and generally may include a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robot arms 1002, 1003 in a first operating mode.

Each of the robot arms 1002, 1003 may include a plurality of members, which are connected through joints, and an attaching device 1009, 1011, to which may be attached, for example, a surgical tool “ST” supporting an end effector 1100, in accordance with any one of several embodiments disclosed herein, as will be described in greater detail below.

Robot arms 1002, 1003 may be driven by electric drives (not shown) that are connected to control device 1004. Control device 1004 (e.g., a computer) may be set up to activate the drives, in particular by means of a computer program, in such a way that robot arms 1002, 1003, their attaching devices 1009, 1011 and thus surgical instrument 10 (including end effector 300) execute a desired movement according to a movement defined by means of manual input devices 1007, 1008. Control device 1004 may also be set up in such a way that it regulates the movement of robot arms 1002, 1003 and/or of the drives.

Medical work station 1000 may be configured for use on a patient 1013 lying on a patient table 1012 to be treated in a minimally invasive manner by means of end effector 1100. Medical work station 1000 may also include more than two robot arms 1002, 1003, the additional robot arms likewise being connected to control device 1004 and being telemanipulatable by means of operating console 1005. A medical instrument or surgical tool (including an end effector 1100) may also be attached to the additional robot arm. Medical work station 1000 may include a database 1014, in particular coupled to with control device 1004, in which are stored, for example, pre-operative data from patient/living being 1013 and/or anatomical atlases.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A cooling assembly for use with an electrosurgical instrument, the cooling assembly comprising:
a body, at least a portion of an inner perimeter of the body configured for selective engagement with a jaw member of the electrosurgical instrument; and
a cavity defined within the body and configured to contain a fluid therein capable of providing at least one of cooling or thermal insulation to areas laterally adjacent the jaw member.

2. The cooling assembly according to claim 1, wherein the fluid is passively cooled.

3. The cooling assembly according to claim 1, wherein the fluid is actively cooled.

4. The cooling assembly according to claim 1, wherein the fluid includes at least one of expandable gas and alcohol.

5. The cooling assembly according to claim 1, wherein the body includes silicone.

6. The surgical instrument according to claim 1, wherein the body is U-shaped.

7. The cooling assembly according to claim 1, wherein the fluid is configured to provide at least one of cooling or thermal insulation to areas distally adjacent the jaw member.

8. A surgical instrument, comprising:
a handle assembly;
an elongated shaft extending distally from the handle assembly;
an end effector engaged to a distal portion of the elongated shaft, the end effector including a first jaw member and a second jaw member, at least one of the first and second jaw members movable with respect to the other jaw member to grasp tissue therebetween; and
a cooling assembly disposed in mechanical cooperation with the first jaw member, the cooling assembly including a deployable reservoir configured to house a fluid therein, the cooling assembly configured to provide at least one of cooling or thermal insulation to areas laterally adjacent the first jaw member.

9. The surgical instrument according to claim 8, wherein the cooling assembly is disposed in mechanical cooperation with the second jaw member and is configured to provide at least one of cooling or thermal insulation to areas laterally adjacent the second jaw member.

10. The surgical instrument according to claim 8, wherein the fluid is passively cooled.

11. The surgical instrument according to claim 8, wherein the fluid is actively cooled.

12. The surgical instrument according to claim 8, wherein the fluid includes at least one of expandable gas and alcohol.

13. The surgical instrument according to claim 8, wherein the deployable reservoir is U-shaped.

14. The surgical instrument according to claim 8, wherein the cooling assembly is configured to provide at least one of cooling or thermal insulation to areas distally adjacent the first jaw member.

15. The surgical instrument according to claim 8, wherein a tissue-contacting surface of the first jaw member defines a plane, and wherein an entirety of the deployable reservoir is spaced from the plane.

16. The surgical instrument according to claim 8, wherein the cooling assembly overlaps an intersection of a lateral edge and a tissue-contacting surface of the first jaw member.

17. The surgical instrument according to claim 8, wherein the deployable reservoir is expandable from a first position wherein the deployable reservoir includes a first amount of fluid therein, to a second position wherein the deployable reservoir includes a second amount of fluid therein.

18. The surgical instrument according to claim 17, wherein in the first position, the deployable reservoir is undeployed, wherein in the second position, the deployable reservoir is deployed, and wherein the second amount of fluid is greater than the first amount of fluid.
